(19) United States
(12) Patent Application Publication
(10) Pub. No.: US 2004/0049261 A1
(43) Pub. Date: Mar. 11, 2004

(54) Medical devices

(76) Inventor: Yixin Xu, Newton, MA (US)

Correspondence Address: FISH & RICHARDSON PC, 225 FRANKLIN ST, BOSTON, MA, 02110, US

(21) Appl. No.: 10/237,517

(22) Filed: Sep. 9, 2002

(51) Int. Cl. A61F 2/06

(52) U.S. Cl. 623/1.15

(57) ABSTRACT

An implantable medical device such as a stent includes a molybdenum/rhenium alloy.

### TECHNICAL FIELD

The invention relates to medical devices such as, for example, stents.

### BACKGROUND

The body includes various passageways such as arteries, other blood vessels, and other body lumens. These passageways sometimes become occluded or weakened. For example, the passageways can be occluded by a tumor, restricted by plaque, or weakened by an aneurysm. When this occurs, the passageway can be reopened or reinforced, or even replaced, with a medical endoprosthesis. An endoprosthesis is typically a tubular member that is placed in a lumen in the body. Endoprostheses stents include covered stents, also sometimes called “stent-grafts”.

Endoprostheses can be delivered inside the body by a catheter that supports the endoprosthesis in a compacted or reduced-size form as the endoprosthesis is transported to a desired site. Upon reaching the site, the endoprosthesis is expanded, for example, so that it can contact the walls of the lumen.

The expansion mechanism may include forcing the endoprosthesis to expand radially. For example, the expansion mechanism can include the catheter carrying a balloon, which carries a balloon-expandable endoprosthesis. The balloon can be inflated to deform and to fix the expanded endoprosthesis at a predetermined position in contact with the lumen wall. The balloon can then be deflated, and the catheter withdrawn.

In another delivery technique, the endoprosthesis is formed of an elastic material that can be reversibly compacted and expanded, e.g., elastically or through a material phase transition. During introduction into the body, the endoprosthesis is restrained in a compacted condition. Upon reaching the desired implantation site, the restraint is removed, for example, by retracting a restraining device such as an outer sheath, enabling the endoprosthesis to self-expand by its own internal elastic restoring force.

To support a passageway open, endoprostheses are sometimes made of relatively strong materials, such as stainless steel or Nitinol (a nickel-titanium alloy), formed into struts or wires. These materials, however, can be relatively radiolucent. That is, the materials may not be easily visible under X-ray fluoroscopy, which is a technique used to locate and to monitor the endoprostheses during and after delivery. To enhance their visibility (e.g., by increasing their radiopacity), the endoprostheses can be coated with a relatively radiopaque material, such as gold.

### SUMMARY

In one aspect, the invention features a stent including a molybdenum/rhenium alloy. Preferred molybdenum/rhenium alloys provide the stent with good radiopacity. In addition, preferred molybdenum/rhenium alloys are strong, flexible and have good ductibility.

The molybdenum/rhenium alloy may include, for example, at least 10% molybdenum by weight and at least 10% rhenium by weight. Preferably, the molybdenum/rhenium alloy includes between about 10% and 70% molybdenum by weight and between about 30% and 90%, and more preferably between about 35% and 55%, rhenium by weight. Preferred molybdenum/rhenium alloys have a density of from about 8 and about 19 g/cm³, more preferably between about 10 g/cm³ and 15 g/cm³. The molybdenum/rhenium alloys preferably have a tensile strength of from about 40 ksi to about 300 ksi, more preferably between 130 ksi and 190 ksi, and a modulus of elasticity from about 47,000 ksi to about 67,000 ksi.

In another aspect, the invention features an implantable medical device, for example, an endoprothesis such as a stent or filter, including the molybdenum/rhenium alloy.

In another aspect, the invention features a medical device such as a guidewire or a braided rotating shaft, designed for use into the body, including the molybdenum/rhenium alloy.

In another aspect, the invention features implanting the implantable medical device, or using the medical device designed for use in the body.

Other aspects, features, and advantages of the invention will be apparent from the description of the embodiments thereof, and from the claims.

### DESCRIPTION OF DRAWINGS

FIG. 1 is a perspective view of a stent composed of a molybdenum/rhenium alloy;

FIG. 2 is a perspective view of a composite tubing including a molybdenum/rhenium alloy;

FIG. 3 is a perspective view, in close-up, of a stent partially composed of a molybdenum/rhenium alloy;

FIG. 4 is a schematic view, in close-up, of a stent partially composed of a molybdenum/rhenium alloy; and

FIG. 5 is a schematic view, in close-up, of a stent partially composed of a molybdenum/rhenium alloy.

### DETAILED DESCRIPTION

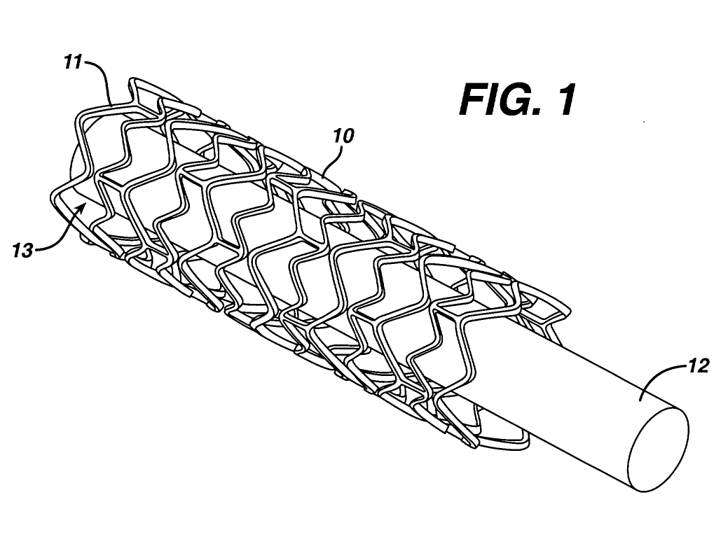

Referring to FIG. 1, a support 12 carries a stent 10, which is the form of a tubular member including struts 11 and openings 13. Depending on the type of stent, support 12 can be a balloon catheter or a catheter shaft.

The stent is composed of a molybdenum/rhenium alloy. The alloy may contain other metals in addition to molybdenum and rhenium, but preferably the alloy consists essentially of molybdenum and rhenium. Molybdenum/rhenium alloy tubing, sheet, foil, and wire is available from Rhenium Alloys, Inc., of 1329 Taylor Street, Elyria, Ohio. An example of a preferred tubing is composed of an alloy including 47.5% by weight rhenium and the balance molybdenum. This alloy has a density of 13.5 g/cm³, a modulus of elasticity of 53,623 ksi, a tensile strength of 123 ksi, and a percent elongation of 22%.

The molybdenum/rhenium alloy may include, for example, between about 30% and 60% or between about 40% and 50% rhenium by weight and, for example, between 40% and 70% or between 50% and 60% molybdenum by weight.

A sheet or foil can be folded and welded to provide a tube using inert gas or electron beam methods, with appropriate protection against oxidation. The tubing can then be drawn or extruded to the desired diameter, or used to fabricate a stent directly. Depending on the application, the stent may have a diameter of between, for example, 1 mm to 46 mm (1 mm to 5 mm for a coronary stent, 20 mm to 46 mm for AAA and TAA stents, in between for peripheral and non-vascular stents). The tubing alternatively can be made, for example, by seamless drawing or extrusion.

After the molybdenum/rhenium alloy tubing has been drawn to the desired diameter, portions of the tubing are removed to provide the strut 11/opening 13 arrangement. The portions can be removed by laser cutting, as described, for example, in U.S. Pat. No. 5,780,807, which is hereby incorporated by reference. Alternatively, the portions can be removed by electrochemical machining, electrical discharge machining, abrasive cutting/grinding methods, or photoetching. Stent 10 can then be finished by electropolishing to a smooth finish, by conventional methods. The stent also can be annealed.

Stent 10 can then be delivered and expanded by generally conventional methods.

Stent 10 can be a part of a stent-graft. The stent-graft can be a stent attached to a biocompatible, non-porous or semi-porous polymer matrix made of polytetrafluoroethylene (PTFE), expanded PTFE, polyethylene, urethane, or polypropylene. Stent 10 can include a releasable therapeutic agent or a pharmaceutically active compound, such as described in U.S. Pat. No. 5,674,242, and commonly-assigned U.S. Ser. No. 09/895,415, filed Jul. 2, 2001, all hereby incorporated by reference. The therapeutic agents or pharmaceutically active compounds can include, for example, anti-thrombogenic agents, antioxidants, anti-inflammatory agents, anesthetic agents, anti-coagulants, and antibiotics.

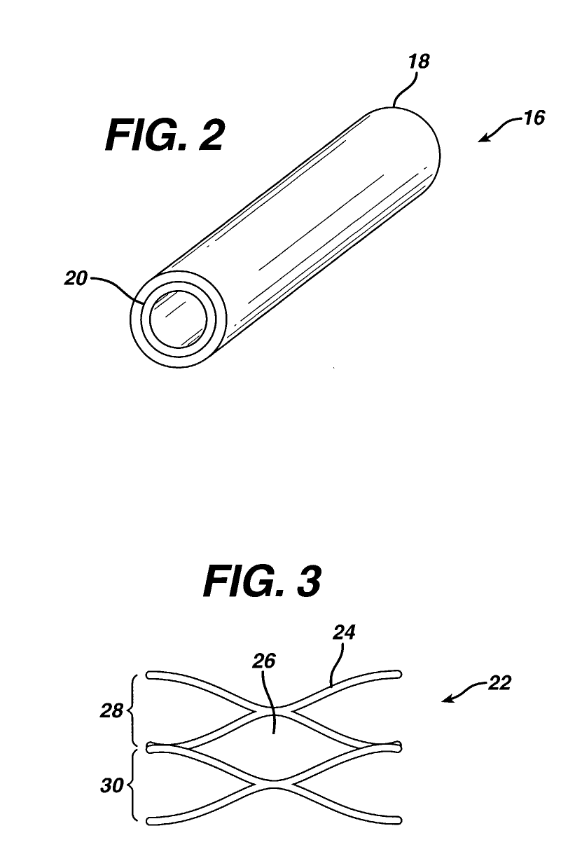

Referring to FIG. 2, a composite tubing 16 includes an outer layer 18 composed of nitinol and an inner layer 20 composed of the molybdenum/rhenium alloy. The composite tubing can be made by co-drawing of two components, by coating one material to another, using CVD (chemical vapor deposition) and PVD (physical vapor deposition) method, or by electric plating one material to another. Outer layer 18 alternatively can be composed of stainless steel. Tubing 16 can be drawn and converted into a stent (with stents) using the methods described above. The two-layer stent can have a combination of properties fully or in part provided by each layer. Preferred two-layer stents can have, for example, good radiopacity, flexibility, and strength.

The portion of outer layer 18 and inner layer 20 in tube 16 (and in the resulting stent) can be reversed.

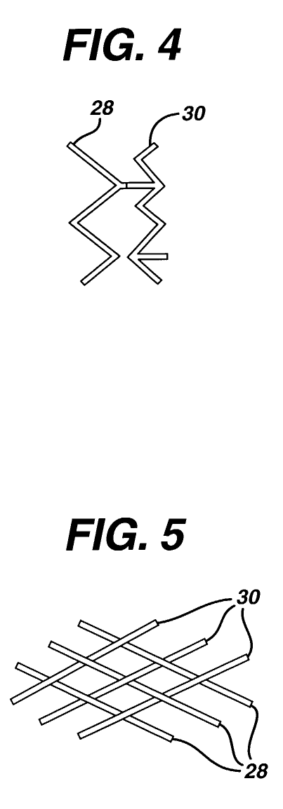

Referring to FIG. 3, an alternative embodiment of a stent 22 includes struts 24, openings 26, and portions 28 and 30. Portion 28 is composed of the molybdenum/rhenium alloy and portion 30 is composed of, for example, nitinol or stainless steel. Portion 28 may be in the form of a band or bands positioned periodically along the length of the stent. Alternatively, portion 28 may be combined with portion 30 as shown in FIGS. 4 and 5. The stent may include, for example, from 10% to 90% or 100% of the molybdenum/rhenium alloy by weight. Molybdenum/rhenium alloy portion(s) 28 can be joined to portion 30 by, for example, welding. Portion 28 provides the stent with enhanced radiopacity. When portion 30 is composed of nitinol or other self-expanding material, the stent will have some portion(s) that are self-expanding and other portion(s) (the molybdenum/rhenium portion(s)) that are balloon expandable.

The molybdenum/rhenium alloy also can be used in other endoprostheses. For example, the molybdenum/rhenium alloy can be used in filters such as removable thrombus filters described in Kim et al., U.S. Pat. No. 6,146,404, which is hereby incorporated by reference; in intravascular filters such as those described in Daniel et al., U.S. Pat. No. 6,171,327, which is hereby incorporated by reference; and vena cava filters such as those described in Soon et al., U.S. Pat. No. 6,342,062, which is hereby incorporated by reference.

The preferred molybdenum/rhenium alloy can also be used in guidewires such as a Meier Steerable Guide Wire (for AAA stent procedure) and an ASAP Automated Biopsy System described in U.S. Pat. Nos. 4,958,625, 5,368,045, and 5,090,419, which are hereby incorporated by reference herein.

Other embodiments are within the claims.

What is claimed is:

1. A stent comprising a tubular member comprising a molybdenum/rhenium alloy.

2. The stent of claim 1, wherein the molybdenum/rhenium alloy includes at least 10% molybdenum by weight and at least 10% rhenium by weight.

3. The stent of claim 2, wherein the molybdenum/rhenium alloy includes at least about 30% rhenium by weight.

4. The stent of claim 1, wherein the molybdenum/rhenium alloy includes between about 10% and about 70% molybdenum by weight and between 30% and about 90% rhenium by weight.

5. The stent of claim 1, wherein the molybdenum/rhenium alloy includes between about 35% and 55% rhenium by weight.

6. The stent of claim 5, wherein the molybdenum/rhenium alloy includes between 45% and 65% molybdenum by weight.

7. The stent of claim 1, wherein the alloy consists essentially of molybdenum and rhenium.

8. The stent of claim 1, wherein the tubular member comprises a first portion comprising the molybdenum/rhenium alloy and a second portion.

9. The stent of claim 8, wherein the second portion comprises a material selected from the group consisting of stainless steel and nickel-titanium alloy.

10. The stent of claim 1, wherein the tubular member comprises a first layer comprising the molybdenum/rhenium alloy and a second layer.

11. The stent of claim 10, wherein the second layer is outside the first layer.

12. The stent of claim 1, wherein the tubular member comprises struts.

13. The stent of claim 8, wherein the tubular member has struts comprising the first portion and struts comprising the second portion.

14. The stent of claim 1, wherein the molybdenum/rhenium alloy has a density of at least 8 g/cm³.

15. The stent of claim 14, wherein the molybdenum/rhenium alloy has a density of less than 19 g/cm³.

16. The stent of claim 1, wherein the molybdenum/rhenium alloy has a tensile strength of from about 40 ksi to about 300 ksi.

17. The stent of claim 1, wherein the molybdenum/rhenium alloy has a modulus of elasticity of between about 47,000 to about 67,000 ksi.

18. An implantable medical device comprising a molybdenum/rhenium alloy.

19. The implantable medical device of claim 18, wherein the device is a filter.

20. The implantable medical device of claim 18, wherein the device is an endoprosthesis.

21. A guidewire for comprising an molybdenum/rhenium alloy.
